(19) United States
(12) Patent Application Publication
(10) Pub. No.: US 2026/0298914 A1
(43) Pub. Date: Oct. 1, 2026

(54) RAPID GLYCAN SAMPLE PREPARATION METHODS, SYSTEM AND KITS

(71) Applicant: Phenomenex, Inc., Torrance, CA (US)

(72) Inventors: Mohammadali Masigol, Rancho Cordova, CA (US); Mastooreh Chamanian, Rancho Palos Verdes, CA (US); Jianli Zhao, Cypress, CA (US); Mihir Mahendrabhai Thakar, Torrance, CA (US)

(21) Appl. No.: 19/478,141

(22) PCT Filed: Apr. 24, 2024
(86) PCT No.: PCT/US2024/026038

§ 371 Date: Oct. 23, 2025

Related U.S. Application Data
(60) Provisional application No. 63/497,916, filed on Apr. 24, 2023.

(51) Int. Cl. G01N 33/543; G01N 1/40

(52) U.S. Cl. CPC G01N 33/54326; G01N 1/4044

(57) ABSTRACT

Disclosed are methods, systems and kits for the separation and recovery of glycans from a glycosylated substrate. Magnetic particles are incorporated in a sample containing the glycosylated substrate. The magnetic particles are utilized to efficiently mix the sample through various steps of the separation and recovery workflow. The magnetic particles are surface 5 functionalized so as to attach released glycans on the surface of the particles. Disclosed systems include a device that is capable of generating an electromagnetic field, wherein the samples containing the magnetic particles are placed for processing.

### PRIORITY

This application is being filed on Apr. 24, 2024 as a PCT International patent application, and claims priority to U.S. Provisional Patent Application No. 63/497,916, filed on Apr. 24, 2023 and entitled Rapid Glycan Samples Preparation Methods, System and Kits. The disclosure of the provisional application is incorporated herein by reference in its entirety.

### FIELD OF INVENTION

The current application relates to methods, systems, and kits for biological sample preparation. More specifically the disclosure relates to the rapid and automatable methods, systems, and kits for glycan sample preparation, i.e., separation and recovery of glycans from glycosylated substrates.

### INTRODUCTION

Glycans are chain-like structures that are composed of single sugar molecules (monosaccharides) linked together by chemical bonds. Glycans are usually found attached to proteins and lipids in living organisms. The glycosylation of proteins is a process in which complex glycans are chemically attached to proteins to form glycoproteins. It has been estimated that more than half of all human proteins are glycosylated. The attached glycans provide proteins with important structural and functional properties. The constituent monosaccharides, their combination and bonding position, and the type of branching can all affect the properties and roles of these glycosylated proteins.

Glycans are important actors in cell-cell interactions and communication, and they also control the behavior of the immune system in many different ways. For example, glycans are one indicator of whether cells or proteins belong to the body. Foreign glycan structures and patterns are recognized by the immune system as “non-self”′ and activate an immune response. Conversely, the presence of “self” glycans can act as a signal to prevent the immune system from over-reacting. Recently, there has been growing interest in the crucial role of glycans as components of antibodies, affecting their binding to pathogens and infected cells and either neutralize them or tag them for removal by white blood cells. Differences in glycan structure can change the target, binding ability and activity of an antibody.

Given their various importance in biological pathways, the study of glycans has become essential in the biopharmaceutical industry, for example with respect to biomarker discovery, cancer drug development, and also in regard to efficacy and safety assessment of protein therapeutics.

Current analytical methods for glycan related studies include the use of capillary electrophoresis and hydrophilic interaction liquid chromatography, and although these can be effective, they require extensive sample preparation, including glycoprotein capture, glycan release, labelling, purification, and pre-concentration steps. It is known in the industry that the current standard methods for glycan sample preparation include numerous steps which require repetitive and laborious human “hands-on” time, including multiple pipetting steps several and sample transfer steps. Additionally, the current protocols are time-consuming and they require a multitude of laboratory equipment, including centrifuges, vacuum hoods, heaters, well plates. During each step a user is required to move the sample from one particular lab equipment or device to another. Furthermore, the current protocols typically require the user to manually mix the samples throughout the various steps by pipetting up and down. This is inefficient and introduces multiple opportunities for human error or sample mishandling. These steps result in protocols which are time consuming and inherently have multiple points of potential human error, affecting the reliability, the robustness and the reproducibility of results, which in turns increases laboratory cost if the protocol needs to be repeated due to errors.

Accordingly, there remains a need for efficient and less error-prone methods of glycan sample preparation. More specifically, there is a need for process which decreases human interaction and reduces “hands-on” time with the sample. There further is a need for a process which introduces efficient yet gentle mixing of the sample and does not rely on crude methods, such as pipetting, which does not result in effective mixing and can damage the protein sample.

Additionally, there remains a need for a system which reduces the required numbers of sample transfer from one piece of equipment to another, thereby reducing the number of devices needed to complete the protocol, and reducing the time and cost associated with each sample preparation.

### SUMMARY

Disclosed herein are methods, systems and kits for improved glycan sample preparation. More specifically, the present disclosure relates to the separation and recovery of glycans from a glycosylated substrate, though the use of magnetic particles mixed incorporated in a sample containing the glycosylated substrate. The magnetic particles are utilized to efficiently mix the sample through various steps during the disclosed workflows. The efficient and gentle mixing provided by the magnetic particles results in increased reaction kinetics and increased reaction yields. The mixing of the particles is actuated by incorporating the sample vessel within a device that is capable of generating a magnetic field, and more specifically an electromagnetic field.

The disclosed methods, systems and kits provide for glycan sample preparation workflows which, when compared to the current benchmark methods in the industry, are less time consuming and reduce potential human error and “hands-on” time. The current methods provide improved reliability, robustness and the reproducibility of results, by decreasing human interaction with samples and by improving the reaction kinetics of protein digestion, labelling of glycans, and attachment and release of glycans from surface of the magnetic particles.

In one aspect, method is presented for the separation and recovery of glycan from a glycosylated substrate. The method includes the steps of:
a) adding an enzymatic agent to a sample containing a glycosylated substrate and releasing glycans from the glycosylated substrate;
b) labelling released glycans;
c) attaching labelled glycans to magnetic particles; and
d) disassociating the labelled glycans from the magnetic particles;
wherein a magnetic field is applied during each of the steps a) through d).

In one embodiment, a further step is conducted of collecting the disassociated labelled glycans. In one embodiment, a glycosylated substrate is prepared in a sample prior to adding the enzymatic agent, wherein the preparation optionally includes a denaturing step prior to the introduction of an enzymatic agent. The denaturing step can comprise a variety of known techniques and workflows for the denaturing a glycosylated substrate, such as a protein. For example in various embodiments the glycosylated substrate can be denatured by the addition of heat to the sample, through mechanical agitation, or by the addition of a denaturing agent (which will likely be specific to the substrate/protein of interest), or through a combination of any of these known techniques.

In accordance with some aspects, a glycosylated substrate refers to a glycoconjugate, a glycoprotein, a glycolipid, a glycopeptide, peptidoglycans, glycosides, an antibody, an antibody-drug conjugate, and/or lipopolysaccharides, or any molecule or compound having a glycan structure attached thereon.

In other aspects, a system is disclosed for the separation and recovery of glycans from a glycosylated substrate. The system includes a device capable of generating a magnetic field, more specifically, an electromagnetic field. The device can generate an electromagnetic field for purposes of mixing samples contained therein, through the incorporation of the magnetic particles contained in the sample. The device can, therefore, also be referred to as an electromagnetic mixer.

The device of the disclosed systems can generate a changing magnetic field and may utilize oscillating electromagnetic fields to fully disaggregate magnetic particles, allowing an optimal exposure and enhanced mixing with surrounding solution. Therefore, the reaction kinetics can be significantly improved, in comparison to more traditional mixing means which utilize a mechanical vortex shaker or pipet mixing of samples.

In other aspects of the disclosure, a kit is provided for glycan sample preparation. A kit for the separation and recovery of glycans from a glycosylated substrate, comprises: magnetic particles, at least one enzymatic reagent, at least one labelling reagent; and optionally at least one reagent for attaching and/or disassociating glycans to the plurality of magnetic particles.

The plurality of magnetic particles provided with the kit, are in accordance with the descriptions provided herein, and capable of being used with the devices disclosed herein. The magnetic particles may be provided in a container which is also incorporated in the kit. Further containers can also be provided which are appropriate for use with the device disclosed in the system. The kit may further comprise various buffers and reagents which are utilized for processing of the workflows described herein with respect to glycan separation and recovery from a glycosylated substrate.

### BRIEF DESCRIPTION OF FIGURES

The skilled person in the art will understand that the drawings, described below, are for illustration purposes only. The drawings are not intended to limit the scope of the applicants' teachings in any way.

FIG. 1 results for glycan capture using the workflow, magnetic particles and systems described in embodiments of this disclosure.

FIG. 2 illustrates the sample clean-up efficiency of the disclosed workflows. Graph A shows a chromatograph of glycan containing sample prior to clean-up and Graph B shows a chromatograph of a glycan containing sample after cleanup according to workflows disclosed herein.

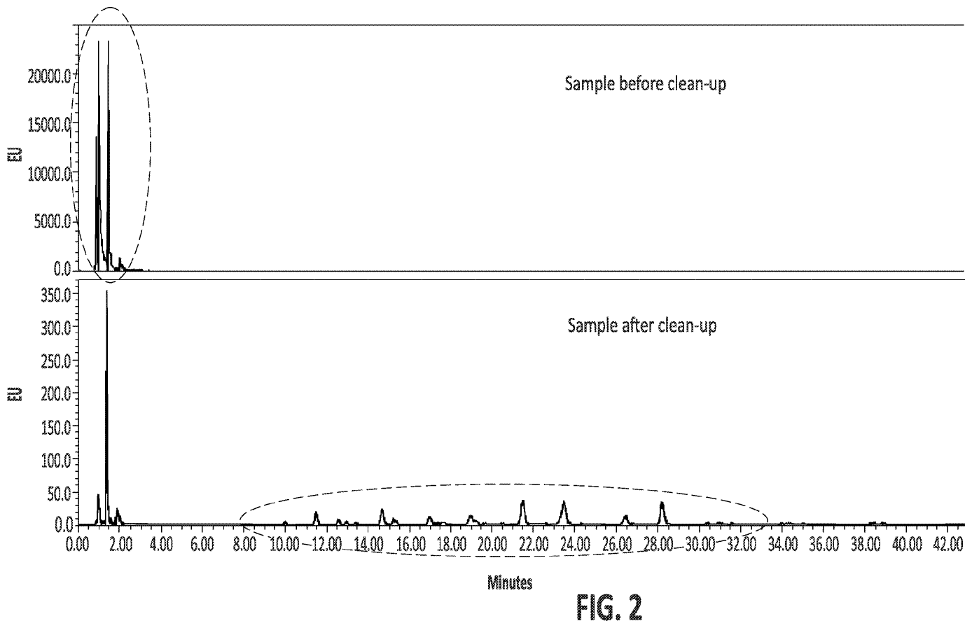

FIG. 3 shows a chromatograph of a sample processed according to the workflows disclosed herein, with magnetic particles having a carboxyl surface functionality.

### DETAILED DESCRIPTION

Disclosed herein are methods for more efficient and automatable glycan sample preparation. More specifically the current disclosure presents methods, systems and kits which are utilized for glycan separation and recovery from a glycosylated substrate. The methods and workflows outlined herein show improved efficiency, can be performed with a substantial reduction of “hands-on” time, and they are achievable through the use of a single laboratory device, without the necessity of multiple equipment sample transfers which lead to opportunities for error and sample mishandling. Thus, the methods disclosed herein provide robustness of results, reliability and reproducibility, all of which are critical characteristics for glycan analysis within the biopharma industry and any laboratory setting where glycan sample preparation is conducted.

### Selected Definitions

The terminology used herein is for the purpose of describing particular embodiments only and is not intended to be limiting of the disclosure.

The singular forms “a”, “an” and “the” are intended to include the plural forms as well, unless the context clearly indicates otherwise.

The term “and/or” or “and or” refers to and encompasses any and all possible combinations of one or more of the associated listed items.

The term “about,” when referring to a measurable value such as length, width, diameter, radius, or an amount of a compound, dose, time, temperature, and the like, is meant to encompass variations of 10%, 5%, 1%, 0.5%, or even 0.1% of the specified amount.

The terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. Unless otherwise defined, all terms, including technical and scientific terms used in the description, have the same meaning as commonly understood by one of ordinary skill in the art to which this disclosure belongs. In the event of conflicting terminology, the present specification is controlling.

All patents, patent applications and publications referred to herein are incorporated by reference in their entirety.

The embodiments described in one aspect of the present disclosure are not limited to the aspect described. The embodiments may also be applied to a different aspect of the disclosure as long as the embodiments do not prevent these aspects of the disclosure from operating for its intended purpose.

The term “ferrimagnetic particles” refers to particles comprising a ferrimagnetic material. Ferrimagnetic particles can respond to an external magnetic field (e.g., a changing magnetic field), but can demagnetize when the external magnetic field is removed. Thus, the ferrimagnetic particles may be efficiently mixed through a sample by external magnetic fields as well as efficiently separated from a sample using a magnet or electromagnet, but can remain suspended without magnetically induced aggregation occurring.

The term “remanence” refers to residual magnetism that a material retains after a magnetic field has been removed. Materials that have a high remanence after the magnetic field has been removed retain a large magnetic field strength, whereas materials that have a low remanence after the magnetic field has been removed have a small magnetic field strength or zero magnetic field strength. The remanence of the magnetic materials may be in a range about 0 emu/g to about 30 emu/g, about 0 emu/g to about 20 emu/g, about 1 emu/g to about 10 emu/g, about 3 emu/g to about 5 emu/g, or less than, equal to, or greater than about 0 emu/g, 1, 2, 3, 4, 5, 6, 7, 8, 9, 10, 11, 12, 13, 14, 15, 16, 17, 18, 19, 20, 21, 22, 23, 24, 25, 26, 27, 28, 29, or about 30 emu/g.

As used herein, the terms “functional group-coated surface” or “surface-functionality” refer to a surface which is coated with moieties which each have a free functional group which is bound to the magnetic particle; as a result, the surfaces of the magnetic particles are coated with the functional group containing moieties. The functional group may be employed to covalently attach a bio-affinity absorbent for biological molecules in solution. In one example, the functional group is a carboxylic acid. A suitable moiety with a free carboxylic acid functional group is a succinic acid moiety in which one of the carboxylic acid groups is bonded to the amine of amino silanes through an amide bond and the second carboxylic acid is a free carboxylic acid group attached or tethered to the surface of the magnetic particle. The functional group-coated surface may be selected from carboxyl groups, amide groups, amino groups, hydroxyl groups, thiol groups, tosyl groups, epoxy groups, alkyl groups, vinyl groups, aryl, or silica groups.

As used herein, the term “glycosylated substrate” refers to structures or molecules which contain proteins covalently bonded with carbohydrate molecules at several specific amino acid residues by the mechanism of glycosylation. Glycosylated substrates can include glycoconjugates, a glycoprotein, a glycolipid, a glycopeptide, peptidoglycans, glycosides, an antibody, an antibody-drug conjugate, proteoglycan, glycosphingolipid, chondroitin sulfate, heparan sulfate, hyaluronan, glycoseaminoglycan, fusion glycoproteins, lipopolysaccharides, or any molecule or compound having a glycan structure attached thereon.

As used herein, the term “glycan(s)” refers to chain-like structures that are composed of single sugar molecules (monosaccharides) linked together by chemical bonds.

### Methods

In certain embodiments, a method is presented for the separation and recovery of glycan from a glycosylated substrate. The method includes the steps of:
a) adding an enzymatic agent to a sample containing a glycosylated substrate and releasing glycans from the glycosylated substrate;
b) labelling released glycans;
c) attaching labelled glycans to magnetic particles;
d) disassociating the labelled glycans from the magnetic particles; and
wherein a magnetic field is applied during each of the steps a) through d).

In one embodiment, a further step is conducted comprising collecting the disassociated labelled glycans. In embodiments, a glycosylated substrate is prepared in a sample prior to adding the enzymatic agent, wherein the preparation optionally includes a denaturing step prior to the introduction of an enzymatic agent. The denaturing step can comprise a variety of known techniques and workflows for the denaturing a glycosylated substrate, such as a protein. For example in various embodiments the glycosylated substrate can be denatured by the addition of heat to the sample, through mechanical agitation, or by the addition of a denaturing agent (which will likely be specific to the substrate/protein of interest), or through a combination of any of these known techniques.

In accordance with some aspects, a glycosylated substrate refers to a glycoconjugate, a glycoprotein, a glycolipid, a glycopeptide, peptidoglycans, glycosides, an antibody, an antibody-drug conjugate, and/or lipopolysaccharides, or any molecule or compound having a glycan structure attached thereon.

In a preferred embodiment the glycosylated substrate undergoes a denaturing step, by heating the sample and mixing or agitating the sample. The temperature of the sample during this step can be in the range of room temperature to 100° C., or preferably about 55° C. to 75° C., or even more preferably 65° C. The duration of this step will depend on the temperature chosen, with higher temperatures requiring less time, and lower temperatures requiring a longer reaction time, as will be understood by one skilled in the art. At a temperature of 65° C., the sample duration of the denaturing step can be about 10 minutes. This step is optional, although it is recommended, because it unfolds the substrate (e.g., protein) so that the sites for glycan cleavage can be more readily available and exposed for the enzymatic agent which will be introduced into the sample in the subsequent step. In theory, conducting the denaturing step should yield a higher amount of glycans once the protocol is completed because more sites on the protein are exposed for cleavage, and hence more glycans can be separated from the substrate, and ultimately recovered during further steps in the workflow/method.

The disclosed methods or workflows rely on the incorporation of a plurality of magnetic particles, which are placed in the sample vessel (e.g., tube, well or well-plate). The magnetic particles are utilized to efficiently mix the sample throughout each step of the workflow. To accomplish this, the sample, having the magnetic particles therein, is placed in a device which is capable of generating a magnetic field, more specifically, an electromagnetic field. The electromagnetic field which is generated by the device can have various characteristics, for example, it can be rotational or static. This means that it can cause the magnetic particles to move in x, y or z direction and stay in movement (rotational) so as to effectuate highly efficient mixing of the sample and reagents therein, or it can cause the particles to gather on the side of the tube and not be in motion (static), which allows for separation of the sample component(s) and the particles, in instances where the sample component(s) need to be removed from the sample vessel. Further details and aspects pertaining to the device and the magnetic particles will disclosed in detail in later sections of the disclosure.

In one embodiment, steps a) through e) outlined above can all occur in the same sample vessel, without the necessity of sample transfer. Meaning that the sample, at any point during the various steps of the workflow, does not need to be taken out of the original sample vessel, and can remain there through the addition of buffers and reagents, up until the collection step where the glycan containing solution is removed from the tube.

Once the glycosylated substrate in the sample has been denatured, then an enzymatic agent is added to the sample, which will react with the substrate at specific sites of attachment and be able to cleave the glycosidic bonds, to release or detach glycans from the glycosylated substrate. After the protein denaturing is complete, a static magnetic force is applied in the device where the sample resides, this causes the magnetic particles to be drawn to the wall of the sample vessel and no longer be dispersed and moving within the sample. This effectively causes the mixing of the sample and particles to cease, and the enzymatic agent can be added into the tube. Thereafter, the rotational magnetic field is again activated so as to force the magnetic particles back into mixing mode. This step can proceed from about 1 minute to 24 hours, and occur at a temperature of ranging from room temperature to 100° C. More preferably the mixing of the enzymatic agent along with the sample will proceed for about 15 minutes, at a temperature of 50° C.

In current methods known in the art, the reactions and molecule interactions occurring in the sample during the denaturing and enzymatic cleavage steps would typically occur on a vortex shaker, which mechanically shakes the entire vessel (typically a 96 well-plate). This type of mixing of the vessel is inferior to the present methods which utilize the magnetic particles within the sample itself to actuate highly efficient mixing of the sample. The more efficient the mixing of the species within the sample, the more kinetic energy and forces are generated which yields in increased species interaction and increased reaction yields. This in turn increases glycan cleavage and amount of released glycans which can be separated and retrieved from the substrate.

In some aspects, the enzymatic agent is chosen from enzymatic Peptide-N-Glycosidase F (PNGaseF), Endoglycosidase H (EndoH), Endo-α-N-Acetylgalactosaminidase, α-Mannosidase, or O-Glycosidase or a combination thereof. The type of reagent chosen will cleave a specific type of glycosidic bond, for example an N-glycan or an O-glycan. “O” linked glycans are attached to an oxygen atom of an amino acid residue in a protein, whereas “N” linked glycans are attached to an amide nitrogen of an asparagine residue of a protein. It will readily understood by those skilled in the art that different enzymes can be used to cleave the various types of glycosidic linkages specific to a protein of interest, whether that be N-glycan, O-glycan, C-glycan or any other type of known linkage. In a preferred embodiment the enzymatic agent is Peptide-N-Glycosidase F (PNGaseF), which can cleave N-glycans.

Once the enzymatic digestion of the glycosylated substrate has occurred, a static magnetic field is again actuated so as to gather the magnetic particles away from the mixing mode and into a location on the wall of the vessel where they are no longer in motion. The labelling step can then be initiated by the addition a dye reagent, which will result in labelling the released glycans for further analysis in later processes, such as capillary electrophoresis, mass spectrometry (MS), or the like. The labelling reagent, or dye, is added while the magnetic beads are in a static mode, and once the reagent has been added to the vessel, the rotational filed is again turned on in the device so that efficient mixing of the released glycans and the labelling reagent can occur, thereby yielding labelled glycans.

The duration of the labelling step will depend on the chemistry and reaction kinetics of the type of labelling reagent chosen. In some aspects the labelling dyes to be used can be chosen from 2-anthranilic acid (2-AA), 2-aminobenzamide (2-AB), 1-aminopyrene-3,6,8-trisulfonic acid (APTS), 8-aminonaphthalene-1,3,6-trisulfonic acid (ANTS), or dye containing a rapid tagging functional group such as N-hydroxysuccinimide carbamate, or a combination thereof. In preferred embodiments, the labelling reagent is 2-aminobenzamide (2-AB). The labelling step can proceed for a duration of 1 min to 24 hours, at a temperature ranging from room temperature to 100° C. More preferably the labelling of the released glycans is allowed to proceed for about 60 minutes, at a temperature of 65° C.

Although the labelling of the released glycans can be conducted at various times or steps throughout the workflow, such as for example once the released glycans have already been attached to the magnetic particles, it is a preferred that the labelling occurs prior to the attachment of the glycans to the particles. In this preferred embodiment, it is believed that a higher labelling reaction or efficiency is achieved since more reaction sites are available if the glycans are free in solution instead of being attached to the particles.

Next the labelled glycans are attached to the magnetic particles. This is accomplished by the addition of a dilution reagent into the sample vessel containing the labelled glycans. Again a static magnetic force is applied within the device so as to draw down the magnetic particles away from the sample solution, and the reagent is added which will effectuate the labelled glycans to attach onto the surface of the magnetic particles. Once the reagent has been added, a rational magnetic field is turned on for efficient mixing and attachment of the labelled glycans onto the particles. This reaction can be proceed for a duration of about 1 to 100 minutes, or 2 to 30 minutes, or 3 to 15 minutes, or 4 to 8 minutes, or more preferably about 5 minutes. In one embodiment the reagent used is acetonitrile (ACN).

The magnetic particles can include surface functionality, meaning that they have specific reactive groups on the surface which can be used to bind the glycans in solution onto the surface of the particle. In one embodiment, the particles have been functionalized with carboxyl groups on the surface. It is believed that the attachment mechanism of glycans with the surface groups of the particles proceeds through a hydrophilic interaction and is not driven by charge attraction.

Once attachment of glycans on the surface of the particles has been achieved, then a static magnetic field is applied to draw down (i.e., cease mixing mode and accumulate the particles together in one location within the vessel, so they are no longer dispersed) the magnetic particles and remove the supernatant solution in the sample vessel. The supernatant at this stage will contain various undesired species which must be removed prior to the sample being processed in later steps for analytical analysis. The supernatant will likely include protein segments, unreacted dye, reagent remnants from prior steps, buffers and so on, which will effect sample integrity if not removed. After this clean-up step is complete, then the labelled glycans which are attached on the surface of the magnetic particles, can be dissociated from the particles, in other words released.

In order to disassociate the attached labelled glycans from the surface of the magnetic particles, an eluent reagent (for example water) is added to the sample vessel and a rotational magnetic field is actuated to start efficient mixing of the particles with the eluent. This step can be repeated several times, for example it may be desired to repeat the step at least two times in mixing mode, to ensure that all the attached labelled glycans are dissociated from the surface of the particles and are now in solution. This ensures effective release of any remaining glycans still attached and can increase the final yield of glycan recovery. A washing step can also be conducted prior to elution of the glycans. Acetonitrile buffer can be added at least once and mixing mode is actuated, so as to wash any remaining unwanted species from the sample. This step is preferably repeated multiple times, for example three times.

The supernatant that results after the dissociation step (i.e. elution step) is then collected, concentrated or otherwise prepared for further analysis. To achieve this, the static magnetic field is turned on to draw down the particles and the supernatant can be removed from the sample vessel.

The sample containing the dissociated labelled glycans can then be prepared for the desired analysis using the appropriate protocols for that particular type of analytical tool. For example, in one embodiment the glycan sample is further analyzed through capillary electrophoresis, liquid chromatography such as reverse phase liquid chromatography or hydrophilic interaction chromatography (HILIC), mass spectrometry (MS), or nuclear magnetic resonance (NMR), or a combination of these techniques.

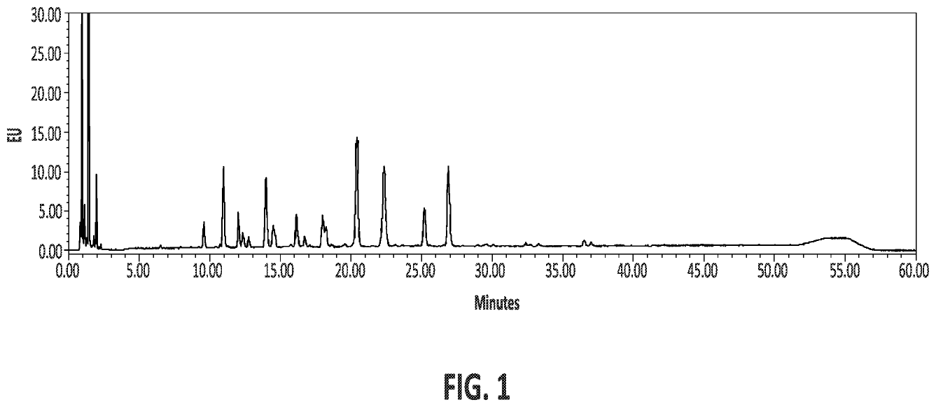

Shown in FIG. 1 is a chromatograph readout of the glycan profile of Aflibercept (Zaltrap) model protein obtained from the above disclosed methods. Hydrophilic interaction liquid chromatography (HILIC) was used to separate 2-AB labeled free N-glycans based on their hydrophobicity, with less polar glycans eluting earlier and more polar glycans eluting later. As can be seen in the chromatograph of FIG. 1, there are well resolved peaks for the major glycan species. The first large peaks on the left are for 2-AB dye and buffer reagent. The following peaks at 10 through 30 minutes include a mix of unbound glycans, neutral and acidic glycans with different levels of sialic acid and/or negative charges, highly sialylated glycans, and fucosylated glycans.

### Systems

Also disclosed are systems for glycan sample preparation. In one embodiment a system for separation and recovery of glycans from a glycosylated substrate is disclosed, the system comprising:
a device capable of generating a magnetic field;
magnetic particles;
at least one enzymatic reagent; and
at least one labelling reagent.

In some embodiments the system further includes reagents for attaching or disassociating glycans to the plurality of magnetic particles. In other embodiments the system further includes at least one denaturing reagent. In further embodiments the system includes buffers, washing solutions, reducing agents, and other reactants which are utilized to carry out the glycan sample preparation workflows disclosed herein.

The device capable of generating a magnetic field, more specifically is capable of generating an electromagnetic field. The device can generate an electromagnetic field for purposes of mixing samples contained therein, through the incorporation of magnetic particles contained in the sample. The device can, therefore, also be referred to as an electromagnetic mixer. For purposes of the following disclosure, “the device” or “electromagnetic mixer” or “magnetic assembly” are used interchangeably and refer to the same component of the disclosed systems.

The device of the disclosed systems can generate a changing magnetic field and may utilize oscillating electromagnetic fields to fully disaggregate magnetic particles, allowing an optimal exposure and enhanced mixing with surrounding solution. Therefore, the reaction kinetics can be significantly improved, in comparison to more traditional mixing means which utilize a mechanical vortex shaker or pipet mixing of samples. This reduces sample transfers and also reduces hands-on time and human interference or potential contamination of samples and sample loss. The use of such a device not only provides substantially more efficient mixing means, which increases reaction kinetics, but also provides means for an automatable sample workflow.

Suitable such devices (magnetic assemblies or electromagnetic mixers) for processing fluid samples are described in WO 2017093896, US 2018-0369831, US 2020-0011773, WO 2020016854, Arnold et al., US20200011773, Arnold et al., U.S. Pat. No. 10,656,147, Campbell et al., and WO 2021203005, Corpstein et al., each of which is incorporated by reference herein in its entirety.

In various aspects, the disclosed system may include a magnetic assembly that includes a plurality of magnetic structures configured to generate a magnetic field gradient within a chamber or plurality of chambers where a fluid/sample containing vessel is placed. The magnetic structures may be formed as a plurality of electromagnets configured to be individually actuated by a controller. Each of the electromagnets may generate a magnetic field within the fluid container. The electromagnets may be differentially actuated to create a magnetic field gradient within the fluid container to agitate, mix, or otherwise influence magnetic particles disposed within the fluid container. Activation of the electromagnets of an electromagnetic structure may generate a magnetic field gradient that influences magnetic particles in an x-y direction. In addition, activation of the electromagnets of a plurality of electromagnetic structures may generate magnetic field gradients that influences magnetic particles in an x-y direction and z-direction. This can be characterized as generating a rotational magnetic field which is utilized during mixing steps of the disclosed glycan sample preparation methods and workflows.

The magnetic particles can be placed inside the sample vessel and dispersed within the fluid, can be configured to be agitated under the effect of magnetic fields (or gradients) generated by a magnetic assembly arranged adjacent to the fluid chambers (e.g., arranged about the periphery chamber where the sample vessel is placed) so as to facilitate the movement of the magnetic particles within the fluid. The magnetic assembly can include a one or a plurality of magnetic structures arranged in horizontal or substantially horizontal layers. Each of the magnetic structures can be formed by one or more magnets, such as an electromagnet. The vertical position of one or more of the magnetic structures, relative to the fluid, can be movable or adjustable, for instance, before, during, or after facilitating the movement of the magnetic particles within the fluid. Adjustment of the vertical position of the one or more of the magnetic structures before facilitating movement of the magnetic particles can be used, for example, to process different sample volumes and/or to affect a characteristic of a magnetic field generated by the magnetic assembly. Vertical movement of the magnetic structures while facilitating the movement of the magnetic particles may add, for example, a vertical component of movement in the particles to provide a more effective or efficient mixing of the particles in the fluid.

Additionally, or alternatively, the electrodes of the various magnetic structures (e.g., of the different vertically-spaced layers) can be selectively energized so as to process different sample volumes and/or to affect a characteristic of a magnetic field generated by the magnetic assembly.

The magnetic assembly structures can be formed from a plurality of electromagnets disposed around the fluid chamber at one or more different vertical heights, with each electromagnet being individually controlled to generate a desired magnetic field within the fluid chamber effective to influence the magnetic particles disposed therein. Based on the selective application of electrical signals to the plurality of electromagnets surrounding the fluid chamber, the magnetic particles can be influenced to rotate, spin, move horizontally side-to-side, and/or vertically up-and-down, or any combination of such movements, within the fluid sample by the combined effect of the magnetic field gradients generated by the various electromagnets.

By way of example, the signals applied to the electromagnets of each magnetic structure (e.g., in a single horizontal layer) can be configured to generate magnetic field gradients substantially in the x-y plane, while the signals applied to the electromagnets of the different magnetic structures, if present (e.g., the electromagnets in different horizontal layers) can result in magnetic field gradients exhibiting a z-direction or vertical component, in this manner, the combined effect of the plurality of electromagnets can produce a magnetic field within a sample container with different characteristics, such as different strengths and/or directionality so as to rapidly and efficiently mix the fluid and/or capture target analytes within the fluid, by way of non-limiting example.

In accordance with one aspect of the disclosure, by applying alternating current (AC) with different phase delays to various electromagnets in the array, all wells can be activated simultaneously, creating a homogenous suspension of magnetic particles in each sample vessel contained within the chamber, reducing diffusion distances to a minimum and improving reaction kinetics. During any buffer exchange process, a steady state magnetic field (DC) may be used to trap beads at corner walls of the sample vessel (this is also characterized as a static magnetic force). The system also may include a temperature control module, a means for heating the samples, and include several temperature sensors, and a cooling fan.

The effective mixing for a high-volume sample (>1 mL) is challenging by mechanical agitation, since the classical two-dimensional shaking cannot bring particles to the top portion of the solution, and the extensive vertical shaking would cause the problem of solution splash and droplets attaching to the vial cap/cover. The effective and homogeneous three-dimensional particle mixing for a high sample volume can be achieved by a two-layer electromagnets structure.

For a broad assay coverage, the system is designed to be compatible with both high magnetic response particles (e.g., ferrimagnetic beads), and low response particles (e.g. superparamagnetic beads) by tuning the AC waveforms applying to the electromagnets set. The power consumption even for a sample containing low-response superparamagnetic beads is less than 5 W.

The magnetic field strength to be applied, the type of magnetic field (rotational or static), the temperature, the time sequence and other parameters are all selected and adjustable through a user interface on the device. Different signal phases controlling each electromagnet may be employed to achieve the optimal suspension of magnetic particles. The system may be extended from a single sample vessel controlled by 4 electromagnets up to 96 wells (sample vessels) controlled by 117 electromagnets with each electromagnet shared by four wells.

### Magnetic Particles

Magnetic beads have been widely used for sample preparations. However, the magnetic property of particles is typically utilized only during the buffer exchange (supernatant removal) process and not during a mixing step as is the case of the present disclosure. The sample mixing with surface-functionalized beads is still typically achieved by mechanical agitation (e.g. shaking, pipette mixing) in prior known workflows. With these types of traditional methods, magnetic particles may aggregate and cluster in discrete areas close to the walls of the container, greatly reducing mixing efficiency.

Suitable magnetic particles for use in the systems and methods described herein include, but are not limited to paramagnetic particles, such as AMPure XP beads available from Beckman Coulter, Inc., Brea, CA. Suitable magnetic particles also include those described in U.S. Pat. Nos. 5,705,628; 5,898,071; and 6,534,262, and in Published PCT Appl. No. WO 2020/018919, published Jan. 23, 2020, US 2021/0139953, published May 313, 2021, all of which are incorporated by reference as if fully set forth herein.

In some aspects the magnetic particles can be ferrimagnetic, meaning that they are comprised of a ferrimagnetic material. Ferrimagnetic particles can respond to an external magnetic field (e.g., a changing magnetic field), but can demagnetize when the external magnetic field is removed. Thus, the ferrimagnetic particles are efficiently mixed through a sample by external magnetic fields as well as efficiently separated from a sample using a magnet or electromagnet but can remain suspended without magnetically induced aggregation occurring.

In some aspects, the magnetic particles described herein are sufficiently responsive to magnetic fields such that they can be efficiently moved through a sample. In general, the range of the field intensity could be the same range as any electromagnet as long as it is able to move the particles. For example, the magnetic field has an intensity of between about 10 mT and about 250 mT, between about 20 mT and about 80 mT, and between about 30 mT and about 50 mT.

In some examples, more powerful electromagnets can be used to mix less responsive microparticles. In some examples, the magnetic field can be focused into the sample as much as possible. Also, the electromagnets can be as close to the sample as possible since the strength of the magnetic field decreases as the square of the distance.

The magnetic particles can be a variety of shapes, which can be regular or irregular. In some examples, the shape maximizes the surface areas of the particles. For example, the magnetic particles can be spherical, bar shaped, elliptical, or any other suitable shape. The magnetic particles can be a variety of densities, which can be determined by the composition of the core. In some examples, the density of the magnetic particles can be adjusted with a coating.

The ferrimagnetic particle may comprise a ferrite. A ferrite includes a ceramic material that comprise an oxide of iron in combination with inorganic compounds of metal, non-metal, or metalloid atoms. For example, a ferrite can comprise iron (III) oxide (Fe₂O₃) blended with one or more additional metallic elements, such as barium, manganese, nickel, zinc, titanium, or any other suitable metallic element. Other examples of ferrites include Fe₂TiO₂, FeTiO₂, MnFeO₄, NiFe₂O₄, MgFe₂O₄. Further examples of ferrites include an iron core including a sulfide or an oxyhydroxide such as FCTSS, Fe₃S₄, FeS, or FeOOH.

Magnetite (Fe₃O₄) is an example of a magnetic material useful in the examples described herein that is an example of a ferrite. Magnetite contains both Fe²⁺ and Fe³⁺ ions. In some cases, the electron spins of the Fe²⁺ and Fe³⁺ ions can be coupled in a crystalline structure such that the magnetite is ferrimagnetic, as described herein. However, in some examples, ferrimagnetic particles comprise any ferrimagnetic material (e.g., ferrite). According to some examples, the ferrimagnetic material (e.g., ferrite) may not be magnetite (Fe₃O₄), however in some examples, magnetite is a suitable ferrimagnetic material.

Ferrites can be categorized into two main families (hard ferrite and soft ferrites) based on their magnetic coercivity (e.g., the material's ability to withstand an external magnetic field without becoming demagnetized).

Hard ferrites have a high magnetic coercivity as well as a high remanence after magnetization. Hard ferrites can be used to make permanent magnets, as hard ferrites do not demagnetize easily in the absence of an external magnetic field, as they can have a high remanence. Examples of hard ferrites include strontium ferrite and barium ferrite.

Soft ferrites have a low magnetic coercivity. Soft ferrites also have a low remanence after magnetization. The magnetization of soft ferrites is easier to change than hard ferrites. Further, the magnetization of soft ferrites can easily reverse direction without dissipating large amounts of energy (e.g., via hysteresis losses). Soft ferrites can also have a high electrical resistivity, thus preventing the formation of eddy currents in the material, which is another source of energy loss.

Soft ferrites can include manganese-zinc (MnZn) ferrite and nickel-zinc (NiZn) ferrite. Thus, in some examples the ferrimagnetic particles comprise MnZn ferrite. In other examples, the ferrimagnetic particles comprise NiZn ferrite. Ferrimagnetic particles comprising MnZn ferrite and/or NiZn ferrite can become magnetized in the presence of an external magnetic field, and thus are able to be moved in the presence of the external magnetic field, but do not significantly aggregate due to magnetically induced aggregation after the external magnetic field is removed, since they have a low remanence.

Some ferrites can be considered to be semi-hard ferrites. Semi-hard ferrites have properties that are between the properties of soft ferrites and the properties of hard ferrites. For example, cobalt ferrite (CoFe₂O₄) is a semi-hard ferrite, which can be magnetized in the presence of an external magnetic field (e.g., a changing magnetic field generated by a magnetic assembly), but does not have a high remanence after the external magnetic field is removed, such that the ferrimagnetic particles comprising a cobalt ferrite core do not significantly aggregate due to magnetically induced aggregation.

The magnetic particles can be a variety of shapes, which can be regular or irregular. In some examples, the shape maximizes the surface areas of the particles. For example, the magnetic particles can be spherical, bar shaped, elliptical, or any other suitable shape. The magnetic particles can be a variety of densities, which can be determined by the composition of the core. In some examples, the density of the magnetic particles can be adjusted with a coating, as described herein.

The strength of the magnetic field may be determined in Gauss. The gauss is the unit of magnetic flux density B in the system of Gaussian units and is equal to Mx/cm2 or g/Bi/s². Gauss is a unit used to measure the strength of a magnetic field and a gaussmeter instrument may be used to make that measurement. The higher the number of Gauss, the more force the magnetic field will have, so the greater the distance will be reached from the surrounding magnet.

The magnetic particle may comprise a magnetic material having a maximum magnetic field strength (Bmax) in a range of from about 20 emu/g to about 250 emu/g, 40 emu/g to 200 emu/g, 50 emu/g to 150 emu/g, or about 80 emu/g to 100 emu/g. The Bmax may be >40, >50, >60, or >70 emu/g. The Bmax may be measured by SQUID (superconducting quantum interference device). The magnetic particle or magnetic bead may have a super high magnetic response, for example, having a Bmax of in a range of about 80-100 emu/g, or about 89 emu/g. This is compared to many commercially available magnetic beads having <40 emu/g.

In some embodiments, the magnetic particle may comprise a magnetic material having a maximum magnetic field strength (Bmax) in a range of from about 20 emu/g to about 250 emu/g and a remanence in a range of from about 0 emu/g to about 30 emu/g.

The magnetic particle may range from about 1 nm mean diameter to about 1 mm mean diameter. In some examples, the magnetic particles may have a mean diameter in a range of 50 nm to 500 nm. In some examples, the magnetic particles may have a mean diameter in a range of 100 to 200 nm. The magnetic bead may have a diameter in a range of about 80 to about 120 nm, or about 100 nm.

The magnetic particles can be substantially solid or can have some degree of porosity. Where the magnetic particles do include some degree of porosity, a pore size of the individual pores can be in a range of from about 5 Å to about 1000 A, about 50 Å to about 500 A. At least a plurality of the pores can be through pores (e.g., extending fully between opposed surfaces). The pore sizes or total porosity of the magnetic particles can be determined according to many suitable methods. For example, the bulk volume of an ideal (e.g., non-porous) magnetic particle can be determined and then the volume of the actual porous skeletal material can be determined. The porosity is then calculated by subtracting the volume of the actual porous skeletal material from the ideal magnetic particle. The porosity of the magnetic particle or individual pore size can also be determined through optical measurements using a microscope and processing the images to measure the individual pores.

The magnetic particles have sufficient surface area to permit efficient binding of a molecule. In some examples, a surface area of the magnetic particles can be in a range of from about 0.1 m²/g to about 500 m²/g, about 1 m²/g to about 200 m² g, or about 10 m²/g to about 100 m² g. In some embodiments, the magnetic particles or magnetic beads have surface area>5 m²/g, >7 m²/g, or >10 m²/g. The surface area may be measured by Brunauer-Emmett-Teller (BET) surface analysis. Brunauer-Emmett-Teller (BET) surface area analysis provides specific surface area evaluation of materials by nitrogen multilayer adsorption measured as a function of relative pressure using a fully automated analyzer. The technique encompasses external area and pore area evaluations to determine the total specific surface area in m²/g.

The magnetic particles described herein can include several different materials. To the extent that mixtures of materials are present, the total magnetic content of the magnetic particles can constitute at least 50 wt % of the magnetic particle, at least 70 wt % of the magnetic particle, at least 80 wt % of the magnetic particle, at least 90 wt % of the magnetic particle, or even 100 wt % of the magnetic particle.

The magnetic particles can include any of those described herein. The non-magnetic material constituting the balance of the magnetic particles can include any of the coating materials described herein, for example. Non-magnetic material can be used as a coating to encapsulate the magnetic portion of the magnetic particle, they can also be used as a functional component to interact with and bind an analyte of interest. Non-magnetic material can also act as filler component.

The magnetic particle or magnetic bead may be surface functionalized with a carboxyl, amino, hydroxyl, silica, streptavidin, or endopeptidase enzyme moiety. The magnetite particle or magnetic bead may have a magnetite core. The magnetite particle or magnetic bead may have a magnetite core coated with a silica shield layer. The silica shield layer may be attached to a silane linker. The silane linker may be attached to a polymer. The polymer may be surface functionalized with a carboxyl, hydroxyl, silica, amine, amide and a combination of different functionalities, or other known surface functionality. In a specific embodiment, the magnetic bead or magnetic particle has a carboxyl group surface functionality.

The magnetic particle may be coated and/or functionalized by any method known in the art. The coating can be, for example, a polymer layer, or a silica layer. Example polymer layers can include polyethylene, polystyrene, poly methyl methacrylate, polyvinyl alcohol, or any other suitable polymer.

For example, synthesis of core-shell Fe₃O₄ nanoparticles (NPs) may be performed via hydrolysis of tetraethyl orthosilicate (TEOS) in the presence of Fe₃O₄ nanoparticles to provide silica-coated magnetite core-shell particles. For example, Fe₃O₄ NPs may be dispersed in water using an ultrasonic water bath, then mixing with ammonia solution (25 wt % in water) and ethanol. TEOS may be added dropwise into Fe₃O₄ suspension with stirring at room temperature overnight. The product may be separated using an external magnet, washed with water and dried at 50° C. The particles may be characterized by x-ray diffraction (XRD), transmission electron microscopy (TEM), selected area electron diffraction (SAED), and UV-Vis absorption spectra. See WO2020018919, which is incorporated herein by reference in its entirety.

A polymer coated magnetic core may be prepared by, for example, dispersing ~4 g of 100 nm magnetite core in 100 ml water under stirring. 10 mL of acrylic acid is added to the flask under stirring with K₂S₂O₈ to get a uniform suspension. The suspension is heated to 80° C. and stirred for 15 hrs. The suspension is cooled to room temperature and a permanent magnet is used to collect the solids from the suspension. The collected solids are washed with water and dried at 60° C. to provide a poly(acrylic acid) coated magnetic bead.

One example of a type of magnetic bead which can be utilized with the methods, systems and kits of the present disclosure includes a magnetite core surrounded from the inner to outer direction by a silica shield, a silane linker, and a polymer. The polymer may be covalently attached to a surface functionality such as a carboxyl, amino, amide combined with another functionality, or silica group. The surface functionality may be used for binding and capture of glycan released from glycosylated substrate in accordance with the workflows disclosed herein. The magnetic particles may exhibit a high magnetic response, for example, having a Bmax of about 89 emu/g, as compared to <40 emu/g for most commercial magnetic beads. The magnetic particles may exhibit a high surface area of >10 m²/g.

### Kits for Glycan Sample Preparation

In some aspects of this disclosure, kits for glycan s ample preparation are disclosed. A kit for the separation and recovery of glycans from a glycosylated substrate, comprises:
a plurality of magnetic particles;
at least one enzymatic reagent;
at least one labelling reagent; and
optionally at least one reagent for attaching and/or disassociating glycans to the plurality of magnetic particles.

The plurality of magnetic particles provided with the kit, are in accordance with the descriptions provided herein, and incorporated within this section of the disclosure and related embodiments. The magnetic particles may be provided in a container which is also incorporated in the kit. Further containers can also be provided which are appropriate for use with the device disclosed in the system. The kit may further comprise various buffers and reagents which are utilized for processing of the workflows described herein with respect to glycan separation and recovery from a glycosylated substrate.

The at least one enzymatic agent provided in the kit will depend on the type of glycosylated substrate which is of interest and the type of glycosidic linkages prevalent in the glycoprotein of interest. The kit may comprise at least one enzyme or a combination of enzymes. In some aspect the enzymatic agent comprises Peptide-N-Glycosidase F (PNGaseF), Endoglycosidase H (EndoH), Endo-α-N-Acetylgalactosaminidase, α-Mannosidase, or O-Glycosidase, or a combination thereof. In one embodiment the enzymatic agent provided in the kit is Peptide-N-Glycosidase F (PNGaseF).

In some aspects the labelling reagent/dyes to be included in the kit are chosen from 2-anthranilic acid (2-AA), 2-aminobenzamide (2-AB), 1-aminopyrene-3,6,8-trisulfonic acid (APTS), 8-aminonaphthalene-1,3,6-trisulfonic acid (ANTS), or a combination thereof. In preferred embodiments, the labelling reagent is 2-aminobenzamide (2-AB).

Various other buffers and reagents may also be included in the kit which are utilized in the glycan separation and recovery workflows disclosed herein. Some of these buffers, solvents and reagents can include denaturing buffers and/or reagents, acetic acid, DMSO, glycerol, sodium cyanoborohydride, tetrahydrofuran (THF), acetonitrile (ACN) and other dilution and elution buffers known to those skilled in the art.

### EXAMPLES

The following examples illustrate glycan sample preparation methods or workflows in accordance with embodiments described herein.

### Example A

In this example, a silica coated magnetic particle is achieved through the following process steps.

A silica encapsulated magnetite core was prepared by dispersing 20 g of 100 nm magnetite core in 800 ml methanol in a 1 L breaker. The mixture was sonicated for 30 minutes to achieve a uniform suspension. 370 ml of 28% ammonia hydroxide was added into the suspension, which was then stirred for 30 minutes. Following sonication, a liquid mixture including 0.5 ml of tetraethyl orthosilicate and 4.5 ml of methanol was added into the suspension dropwise under further sonication over a time span of 0.5 hrs. Following sonication the beaker was covered and the suspension was continuously stirred for 15 hrs. After stirring the silica encapsulated particles were captured with a permanent magnet. The silica encapsulated particles were then washed 5 times with water. The particles were then dried in an oven at 80° C. for 24 hrs.

### Example B

A carboxylated silica coated magnetic core was prepared by dispersing 4 g of silica coated magnetic core particles prepared according to Example A dispersed with 150 ml toluene in a 500 ml flask under stirring. 20 g of (3-tritheoxysilyl) propylsuccinic anhydride was added under stirring to the flask. Thereafter, 0.2 g imidazole was then added under stirring to produce a uniform suspension. The suspension was refluxed at around 114° C. under stirring, for 15 hrs. Following refluxing, the suspension was cooled to room temperature and a permanent magnet was used to collect solids from the suspension. The solids were washed first with methanol once and then with water 5 times and transferred to a 500 ml flask. 150 ml of 0.1 M acetic acid in water was added to the flask under stirring to get a uniform suspension. The suspension was heated to 90° C. for 15 hrs. The suspension was cooled to room temperature. A permanent magnet was used to collect solids from the suspension. The solids were washed with 5 times water and dried in oven at 60° C. for 15 hrs.

### Example 1

### Denaturing and Deglycosylation of a Glycosylated Substrate

PCR tubes (0.2 mL) were placed into an electromagnetic mixer holder (chamber) and 83.3 μL of 3% carboxylated magnetic particles (prepared as in Example B) were added (50% Glycerol/H2O) to each PCR tube. The electromagnetic mixer was turned on and a desired mixing protocol was selected. The contents of the tubes were mixed for 2 min at room temperature. The mixing function (i.e. rotational magnetic field) on the device was halted and a static magnetic field was applied to remove the particles from mixing mode (i.e. draw down) so that the 50% glycerol/water could be removed from each tube. Subsequently, 100 μL fresh LC-MS grade water was added to each vial and mixing was conducted for 1 min. This washing step was repeated 3 times. A static magnetic field was again applied to remove the magnetic particles from mixing mode, and the water was removed from each vial.

Next, 6 mg/mL of glycosylated substrate, i.e., a glycoprotein (aflibercept, branded as “Zaltrap”) solution were prepared. Water can be used to dilute and reach to the desired concentration. For example, when making 72.9 μL of 6 mg/mL solution, 55.4 μL water is added to 17.5 μL from 25 mg/mL stock. To the tubes is added 16 μL of glycoprotein solution (6 mg/mL). Then 4 μL rapid PNGaseF buffer (5×) is added to each tube. The heater on the device is turned to 65 C and mixing protocol is conducted for 10 minutes. After the 10 minutes, then mixing mode was turned off along with the heater and the sample was allowed to cool down for 5 minutes. Once the sample cooled, then 2 μL of rapid PNGaseF enzyme was added to each vial and mixing was turned on again for 15 min at 50° C.

### Labelling of Released Glycans

While the deglycosylation step is occurring, fresh 2-AB labelling reagent solution was prepared by measuring out 5 mg of 2-AB and transferring it to a 0.5 mL tube. Then adding 130 μL DMSO and 70 μL acetic acid to the tube. The amount of labelling reagent prepared during this step will depend on the number of samples which are being processed at one time. If less labelling reagent is required then less amount of 2-AB, acetic acid and DMSO are used, while keeping the ratio constant, (i.e., 2.5 mg 2-AB with 65 μL DMSO and 35 μL acetic acid).

Next, 5 μL of 1M sodium cyanoborohydride in THF was prepared. 20 μL labelling reagent and 5 μL of 1M sodium cyanoborohydride in THF was added to each sample tube and mixed for at 65° C. for 60 min. The samples are protected from light during this step as the 2-AB labelling dye is light sensitive (the electromagnetic mixer can be covered with aluminum foil during this step). After the labelling reaction has concluded, 150 μL acetonitrile is added to each tube and mixed. The addition of acetonitrile to the tubes causes labelled glycans in solution to attached onto the surface of the magnetic particles through a hydrophilic interaction.

### Clean-Up and Elution

The static magnetic field is applied to draw down the magnetic particles within the solution and ACN and dye solution is removed. Fresh 100 μL acetonitrile was added and mixed for 1 minute. The supernatant is then removed. This step was repeated three times. For elution of the attached glycans, 50 μL H2O was added to the tubes, to elute and dissociate labelled glycans from the surface of the magnetic particles. Mixing is conducted for 3 min, then the supernatant is removed and transferred the supernatant to a separate tube (the sample vial). This elution step was repeated two times. The final sample volume should be 2× 50 μL=100 μL. The samples were then prepared for HPLC analysis. The glycan sample was injected into LC for UHPLC characterization using the following conditions:
Sample: N-glycan analysis of aflibercept
Column: ACQUITY UPLC Glycan BEH Amide Column, 130 Å, 1.7 μm, 2.1 mm×150 mm
Mobile Phase: Buffer A, 50 mM ammonium formate in water, pH 4.4; Buffer B, 100% acetonitrile
Flow rate: 0.4 mL/min
Temperature: 60° C.
FLD detector: excitation 360 nm, emission 428 nm
Injection: 20 μL

Gradient of mobile phase in the column:

| Time (min) | % Buffer A | % Buffer B |
|---|---|---|
| 0 | 80 | 20 |
| 2 | 80 | 20 |
| 2.5 | 75 | 25 |
| 50 | 62 | 38 |
| 52 | 40 | 60 |
| 53.5 | 40 | 60 |
| 55 | 80 | 20 |
| 60 | 80 | 20 |

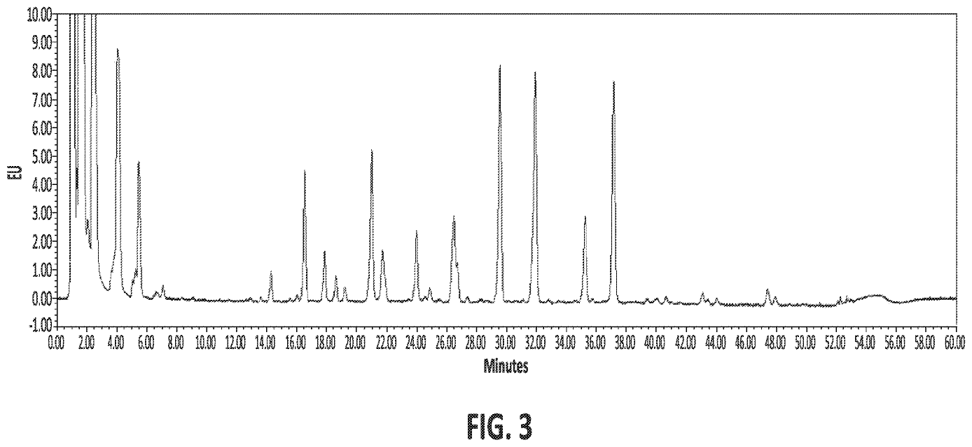

The chromatograph shown in FIG. 3 illustrates the results of the above N-glycan sample preparation workflow of aflibercept according to Example 1, using magnetic particles that have been surface functionalized with carboxyl groups.

The following numbered clauses define further example aspects and features of the present disclosure:
1. A method for recovery of glycans from a glycosylated substrate, the method comprising:
   a) adding an enzymatic agent to a sample containing a glycosylated substrate and releasing glycans from the glycosylated substrate;
   b) labelling released glycans;
   c) attaching labelled glycans to magnetic particles;
   d) disassociating the labelled glycans from the magnetic particles; and
   wherein a magnetic field is applied during each of the steps a) through d).
2. The method of clause 1, further comprising a step of denaturing the glycosylated substrate prior to adding the enzymatic agent.
3. The method of clause 1, further comprising a step of collecting the labelled glycans after step d).
4. The method of clause 1, wherein steps a) through d) occur in the same sample vessel.
5. The method of clause 1, wherein the magnetic particles are present in the sample from step a) through step d).
6. The method of clause 1, wherein the magnetic field is an electromagnetic field.
7. The method of clause 1, wherein the steps a) through d) are conducted in a device capable of generating an electromagnetic field.
8. The method of clause 7, wherein the device comprises a plurality of electromagnetic structures.
9. The method of any one of clauses 1-8, wherein the magnetic field comprises a rotational magnetic field or a static magnetic field.
10. The method of clause 9, wherein the rotational magnetic field is applied in intervals during steps a) through d) for mixing of the sample.
11. The method of clause 9, wherein the static magnetic field is applied at least during steps a), b) and the step of collecting the labelled glycans.
12. The method of any one of clauses 1-11, wherein the glycosylated substrate comprises a glycoconjugate, a glycoprotein, a glycolipid, a glycopeptide, peptidoglycans, glycosides, an antibody, an antibody-drug conjugate, and/or lipopolysaccharides.
13. The method of any one of clauses 1-12, wherein the releasing of glycans from the glycosylated substrate in step a) comprises release of O-glycans, or N-glycans.
14. The method of any one of clauses 1-13, wherein the enzymatic agent comprises Peptide-N-Glycosidase F (PNGaseF), Endoglycosidase H (EndoH), Endo-α-N-Acetylgalactosaminidase, α-Mannosidase, or O-Glycosidase or a combination thereof.
15. The method of any one of clauses 1-14, wherein step b) comprises adding a labelling reagent selected from, 2-anthranilic acid (2-AA), 2-aminobenzamide (2-AB), 1-aminopyrene-3,6,8-trisulfonic acid (APTS), 8-aminonaphthalene-1,3,6-trisulfonic acid (ANTS), or a combination thereof.
16. The method of clause 15, wherein the labelling reagent is 2-aminobenzamide (2-AB).
17. The method of any one of clauses 1-16, wherein step c) comprises adding acetonitrile (ACN) to attach labelled glycans to the magnetic particles.
18. The method of clause 2, wherein the step of denaturing the glycosylated substrate is conducted at a temperature ranging from room temperature to 100° C., for a time duration ranging from 1 min to 24 hours.
19. The method of clause 18, wherein the temperature is about 55° C.-75° C., and the duration is about 5-15 minutes.
20. The method of any one of clauses 1-19, wherein steps a) and b) each are conducted at a temperature range of room temperature to 100° C., for a time duration ranging from 1 min to 24 hours.
21. The method of clause 20, wherein the temperature during step a) is about 40° C.-60° C., and the duration is about 5-25 minutes.
22. The method of clause 20, wherein the temperature during step b) is about 55° C.-75° C., and the duration is about 50-70 minutes.
23. The method of any one of clauses 1-22, wherein steps d) and the step of collecting the labelled glycans are conducted at room temperature for a combined duration of about 5-25 min.
24. The method of any one of clauses 1-23, wherein the magnetic particles are surface-functionalized with carboxyl groups.
25. The method of any one of clauses 1-24, wherein the magnetic particles comprise a polymeric surface coating.
26. The method of any one of clauses 1-25, wherein the magnetic particles are particles having a magnetic strength of about 20 emu/g to 250 emu/g.
27. The method of any one of clauses 1-26, wherein the magnetic particles are ferrimagnetic particles which comprise a ferrite compound.
28. The method of any one of clauses 1-27, wherein step c) comprises the addition of acetonitrile buffer to promote attachment of released glycans to the functional groups on the surface of the magnetic particles.
29. The method of any one of clauses 1-28, wherein step d) comprises adding acetonitrile buffer to promote disassociation of labelled glycans from the surface of the magnetic particles.
30. The method of clause 15, wherein the labelling reagent is 2-anthranilic acid (2-AA).
31. The method of any one of clauses 1-30, further comprising the analysis of the collected labelled glycans through capillary electrophoresis, reverse phase liquid chromatography, hydrophilic interaction chromatography (HILIC), mass spectrometry (MS), or nuclear magnetic resonance (NMR).
32. A system for recovery of glycans from a glycosylated substrate, the system comprising:
   a device capable of generating a magnetic field;
   a plurality of magnetic particles;
   at least one enzymatic agent; and
   at least one labelling reagent.
33. The system of clause 32, wherein the magnetic field is an electromagnetic field.
34. The system of clause 32, wherein the device comprises a plurality of electromagnetic structures.
35. The system of any one of clauses 33-34, wherein the electromagnetic field comprises a rotational field and/or a static field.
36. The system of clause 35, wherein a rotational magnetic field generated by the device actuates movement of the magnetic particles for mixing of a sample placed within the device.
37. The system of clause 35, wherein a static magnetic field generated is actuated to draw down magnetic particles from a sample.
38. The system of clause 32, further comprising at least one reagent for attaching and/or disassociating glycans from the plurality of magnetic particles.
39. The system of any one of clauses 32-38, further comprising a denaturing agent.
40. The system of any one of clauses 32-39, wherein the at least one enzymatic agent comprises Peptide-N-Glycosidase F (PNGaseF), Endoglycosidase H (EndoH), Endo-α-N-Acetylgalactosaminidase, α-Mannosidase, or O-Glycosidase or a combination thereof.
41. The system of any one of clauses 32-40, wherein the at least one labelling reagent comprises 2-anthranilic acid (2-AA), 2-aminobenzamide (2-AB), 1-aminopyrene-3,6,8-trisulfonic acid (APTS), 8-aminonaphthalene-1,3,6-trisulfonic acid (ANTS), or a combination thereof.
42. The system of any one of clauses 32-41, wherein the plurality of magnetic particles are surface-functionalized with carboxyl groups.
43. A kit for the recovery of glycans from a glycosylated substrate, the kit comprising:
   magnetic particles;
   at least one enzymatic reagent;
   at least one labelling reagent; and
   optionally at least one reagent for attaching and/or disassociating glycans to the magnetic particles.
44. The kit of clause 43, wherein the magnetic particles are particles having a magnetic strength of about 20 emu/g to 250 emu/g.
45. The kit of any one of clauses 42-43, wherein the magnetic particles are ferrimagnetic particles comprising a ferrite.
46. The kit of any one of clauses 43-45, wherein the magnetic particles have a mean diameter in a range of 50 nm to 500 nm.
47. The kit of any one of clauses 43-46, wherein the magnetic particles are surface-functionalized with are surface-functionalized with carboxyl groups.
48. The kit of any one of clauses 43-47, wherein the at least one enzymatic agent comprises Peptide-N-Glycosidase F (PNGaseF), Endoglycosidase H (EndoH), Endo-α-N-Acetylgalactosaminidase, α-Mannosidase, or O-Glycosidase or a combination thereof.
49. The kit of any one of clauses 43-48, wherein the at least one labelling reagent comprises 2-anthranilic acid (2-AA), 2-aminobenzamide (2-AB), 1-aminopyrene-3,6,8-trisulfonic acid (APTS), 8-aminonaphthalene-1,3,6-trisulfonic acid (ANTS), or a combination thereof.
50. The kit of any one of clauses 43-49, wherein the at least one reagent for attaching and/or disassociating glycans to the magnetic particles comprises acetonitrile.

It will be appreciated that several of the above-disclosed and other features and functions, or alternatives thereof, can be desirably combined into many other different systems or applications. It will also be appreciated that various presently unforeseen or unanticipated alternatives, modifications, variations or improvements therein can be subsequently made by those skilled in the art which alternatives, variations and improvements are also intended to be encompassed by the following claims.

1. A method for recovery of glycans from a glycosylated substrate, the method comprising:
a) adding an enzymatic agent to a sample containing a glycosylated substrate and releasing glycans from the glycosylated substrate;
b) labelling released glycans;
c) attaching labelled glycans to magnetic particles;
d) disassociating the labelled glycans from the magnetic particles; and
wherein a magnetic field is applied during each of the steps a) through d).

2. The method of claim 1, further comprising a step of denaturing the glycosylated substrate prior to adding the enzymatic agent.

3. The method of claim 1, further comprising a step of collecting the labelled glycans after step d).

4. The method of claim 1, wherein steps a) through d) occur in the same sample vessel.

5. The method of claim 1, wherein the magnetic particles are present in the sample from step a) through step d).

6. The method of claim 1, wherein the magnetic field is an electromagnetic field.

7. The method of claim 1, wherein the steps a) through d) are conducted in a device capable of generating an electromagnetic field.

8. The method of claim 7, wherein the device comprises a plurality of electromagnetic structures.

9. The method of claim 1, wherein the magnetic field comprises a rotational magnetic field or a static magnetic field.

10. The method of claim 9, wherein the rotational magnetic field is applied in intervals during steps a) through d) for mixing of the sample.

11. The method of claim 9, wherein the static magnetic field is applied at least during steps a), b) and the step of collecting the labelled glycans.

12. The method of claim 1, wherein the releasing of glycans from the glycosylated substrate in step a) comprises release of O-glycans, or N-glycans.

13. The method of claim 1, wherein step b) comprises adding a labelling reagent selected from, 2-anthranilic acid (2-AA), 2-aminobenzamide (2-AB), 1-aminopyrene-3,6,8-trisulfonic acid (APTS), 8-aminonaphthalene-1,3,6-trisulfonic acid (ANTS), or a combination thereof.

14. A system for recovery of glycans from a glycosylated substrate, the system comprising:
a device capable of generating a magnetic field;
a plurality of magnetic particles;
at least one enzymatic agent; and
at least one labelling reagent.

15. The system of claim 14, wherein the magnetic field is an electromagnetic field.

16. The system of claim 15, wherein the electromagnetic field comprises a rotational field and/or a static field.

17. The system of claim 14, wherein the device comprises a plurality of electromagnetic structures.

18. The system of claim 14, further comprising at least one reagent for attaching and/or disassociating glycans from the plurality of magnetic particles.

19. The system of claim 14, further comprising a denaturing agent.

20. The system of claim 14, wherein the plurality of magnetic particles are surface-functionalized with carboxyl groups.
